(19) United States
(12) Patent Application Publication
(10) Pub. No.: US 2004/0072862 A1
(43) Pub. Date: Apr. 15, 2004

(54) Compositions for treating ischemia-related neuronal damage

(76) Inventors: Catherine M. Bitler, Menlo Park, CA (US); Paul Wood, Belmont, CA (US); D. Timothy Anstine, Boise, ID (US); Anke Meyer-Franke, Menlo Park, CA (US); Qi Zhao, North Andover, MA (US); Mohamed A. Khan, Mogan Hill, CA (US)

Correspondence Address: PERKINS COIE LLP, P.O. BOX 2168, MENLO PARK, CA, 94026, US

(21) Appl. No.: 10/354,793

(22) Filed: Jan. 29, 2003

Related U.S. Application Data
(63) Continuation-in-part of application No. 09/587,043, filed on Jun. 2, 2000, now Pat. No. 6,541,486.
(60) Provisional application No. 60/137,618, filed on Jun. 4, 1999.
(60) Provisional application No. 60/138,855, filed on Jun. 11, 1999.
(60) Provisional application No. 60/168,256, filed on Nov. 30, 1999.

(51) Int. Cl. A61K 31/4745; A61K 31/4184

(52) U.S. Cl. 514/303; 514/394

(57) ABSTRACT

Compounds, pharmaceutical compositions, and methods of use are described which are effective in inhibiting cell death, particularly apoptotic cell death. The compositions may be used for prevention and treatment of injuries associated with cell death, including ischemia, such as results from stroke or myocardial infarction, trauma, neurodegeneration, and inflammation.

### COMPOSITIONS FOR TREATING ISCHEMIA-RELATED NEURONAL DAMAGE

This application is a continuation-in-part of U.S. patent application Ser. No. 09/587,043, filed Jun. 2, 2000, which is hereby incorporated by reference in its entirety, and which claims priority to U.S. provisional applications having serial Nos. 60/137,618, filed Jun. 4, 1999; 60/138,855, filed Jun. 11, 1999; and 60/168,256, filed Nov. 30, 1999. Each of these applications is also incorporated by reference in its entirety.

### FIELD OF THE INVENTION

The present invention relates to compounds and methods for inhibiting cell death, such as neuronal or myocardial cell death. The compounds and pharmaceutical compositions thereof are particularly effective in inhibiting apoptotic cell death, and thus may be used to protect cells from cell death associated with ischemia, trauma, neurodegeneration, and inflammation.

### References

Barres et al., Neuron 1:791-803 (1988).

Batistatou et al., J. Cell Biol. 122:523-532 (1993).

Brewer et al., J. Neurosci. Res. 35:567-576 (1993).

Busciglio et al., Nature 378:776-779 (1995).

Ghosh et al., Science 263:1618-1623 (1994).

Greenlund et al., Neuron 14:373-376 (1995).

Hinton et al., Arch. Ophthalmol. 116:203-209 (1998).

Kim et al., Science 277:373-376 (1997).

Kirino, T., Brain Res. 239:57-69 (1982).

Koizumi et al., Jpn. J. Stroke 8:1-8 (1986).

Laquis et al., Brain Res. 784:100-104 (1998).

Lazdins et al., J. Exp. Med. 185:81-90 (1997).

Liston et al., Nature 379:349-353 (1996).

MacManus et al., Neurosci. Lett. 164:389-392 (1993).

Meyer-Franke et al., Neuron 15:805-819 (1995).

Nickells, R. W., J. Glaucoma 5(5):345-356 (1996).

Pulsinelli et al., Stroke 10:267-272 (1979).

Schwartz et al., Proc. Natl. Acad. Sci. USA 90 (3):980-984 (1993).

Tamura et al., J. Cereb. Blood Flow Metab. 1:53 (1981).

Vermes et al., J. Immunol. Meth. 184:39-51 (1995).

Vitale et al., Histochemistry 100:223-229 (1993).

Walton et al., Neuroreport 8(18):3871-3875 (1997).

Wyllie et al., J. Pathol. 142:67-77 (1984).

Zhao et al., Brain Res. 649:253-259 (1994).

### BACKGROUND OF THE INVENTION

Apoptosis has been associated with ischemic injury, such as typically occurs in cases of stroke, myocardial infarction, and reperfusion injury (Walton et al., 1997; MacManus et al., 1993). Apoptosis is also associated with immunoreactive and immunodegenerative states and a variety of neurodegenerative disorders. Recent studies on the mechanism of retinal ganglion cell death in experimental glaucoma also indicate that the cells die by apoptosis (Nickells, 1996; Garcia-Valenzuela et al.,1995; Laquis et al., 1998).

Apoptosis is a programmed cell death, occurring in normally functioning human and animal cells when age or state of cell health and condition dictates. It is an active process requiring metabolic activity by the dying cell, and is often characterized by cleavage of the DNA into fragments that give a so called laddering pattern on gels. Cells that die by apoptosis do not usually elicit the inflammatory responses that are associated with necrosis, a passive process in which collapse of internal homeostasis leads to cellular dissolution.

Apoptosis can have particularly devastating consequences when it occurs pathologically in cells that do not normally regenerate, such as neurons. Because such cells are not replaced when they die, their loss can lead to debilitating and sometimes fatal dysfunction of the affected organ.

Various drug strategies have been proposed for treatment of stroke and other neuronal conditions related to ischemia. To date, however, these drugs have been either relatively ineffective or effective only at dosage levels where undesired side effects are observed. For example, anti-coagulants, such as heparin, antivasoconstriction agents, such as flunarazine, excitatory neurotransmitter antagonists, such as MK-801 and AP7, and anti-edemic compounds have shown mixed results, with no clear benefits to outweigh a variety of side effects, including neurotoxicity or increased susceptibility to infection. Verapamil and related compounds, which prevent calcium entry into smooth and striated muscles, appear to be effective only at high drug concentrations, where serious cardiotoxicity effects may ensue. Increased cerebral edema has been observed as a side effect in treatment with dihydropyridines, such as nimodipine. Benzothiazepines, as exemplified by diltiazem, have shown moderate protective effects, but these drugs also appear to cause undesired side effects, such as hypotension, which may be inimical to treatment.

### SUMMARY OF THE INVENTION

In one aspect, the invention provides a pharmaceutical composition, useful for inhibiting cell death, which comprises an effective amount of a compound of formula I, or a pharmaceutically acceptable salt thereof, in a pharmaceutically acceptable carrier.

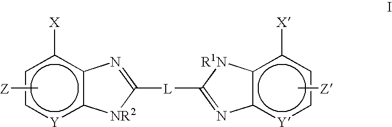

In formula I, X, X′, Z and Z′ are independently selected from the group consisting of hydrogen, lower alkyl, hydroxyl, cyano, carboxylic acid, amino, nitro, and halogen, wherein at least one of X, X′, Z and Z′ is hydroxyl; R¹ and R² are independently selected from hydrogen and lower alkyl; L is —CH₂— or —CHCH₃—; Y is selected from C-Z and nitrogen, and Y′ is selected from C-Z′ and nitrogen. In one embodiment, L is CH₂. In other selected embodiments, R¹ and R² are independently selected from hydrogen and methyl, or R¹ and R² are both hydrogen. In other embodiments, each of Y and Y′ is C—H.

In preferred embodiments, X, X′, Z and Z′ are independently selected from the group consisting of hydrogen, methyl, hydroxyl, cyano, carboxylic acid, amino, nitro, fluoro, and chloro, wherein at least one of X, X′, Z and Z′ is hydroxyl; more preferably, at least one of X and X′ is hydroxyl. In one embodiment, X is hydrogen and X′ is hydroxyl. This includes the embodiment in which the compound is 2-(benzimidazol-2′-yl)methyl-4-hydroxy benzimidazole (where L is CH₂, Y and Y′ are C—H, R¹, R² and X are hydrogen, and X′ is hydroxyl), designated herein as SNX-926, alternatively named 2-(benzimidazol-2-ylmethyl)benzimidazol-7-ol, or a pharmaceutically acceptable salt thereof. This compound is also a feature of the present invention.

In another aspect, the invention provides a method of inhibiting cell death. In accordance with the method, an effective amount of a compound of formula I above, or a pharmaceutically acceptable salt thereof, in a pharmaceutically acceptable carrier, is administered to a subject in need of such treatment. Preferred embodiments of the method include the embodiments of the compound of formula I described above.

In one embodiment of the present method, the cell death being treated or prevented is apoptotic neuronal cell death, such as that associated with stroke, ischemia, neurodegeneration, trauma, an autoimmune response, or inflammation. In another embodiment, the cell death is associated with myocardial damage, such as myocardial infarction and the resulting ischemia, hypoxia and subsequent reperfusion in the affected area, or myocardial damage resulting from therapeutic intervention, e.g. coronary arterial bypass graft (CABG) or percutaneous transluminal coronary angioplasty (PTCA; “balloon” angioplasty).

In a further embodiment of the method, the compounds of formula I are administered in combination with an anti-hypertensive agent, an antibiotic, an immunomodulator, or an anti-inflammatory agent.

Also included within the invention is the methylenebis(benzimidazole) compound, 2-(benzimidazol-2′-yl)methyl-4-hydroxy benzimidazole, noted above.

These and other objects and features of the invention will become more fully apparent when the following detailed description of the invention is read in conjunction with the accompanying drawings.

### BRIEF DESCRIPTION OF THE DRAWINGS

FIGS. 1A-C illustrate synthetic methods useful for preparing symmetrically or unsymmetrically substituted compounds of the invention; and

FIG. 2 shows dose-response data for invention compound SNX-926 in protection of OGD (oxygen-glucose deprived) retinal ganglion cells.

### DETAILED DESCRIPTION OF THE INVENTION

I. Definitions

The terms below have the following meanings unless indicated otherwise.

“Alkyl” refers to a fully saturated acyclic monovalent radical containing carbon and hydrogen, which may be branched or a straight chain. Examples of alkyl groups are methyl, ethyl, n-butyl, n-heptyl, and isopropyl. “Lower alkyl”, a subset of this class, refers to alkyl having one to six carbon atoms, and more preferably one to four carbon atoms.

A “pharmaceutically acceptable salt” of a compound described herein refers to the compound in protonated form with one or more anionic counterions such as chloride, sulfate, phosphate, acetate, succinate, citrate, lactate, maleate, fumarate, palmitate, cholate, glutamate, glutarate, tartrate, stearate, salicylate, methanesulfonate, benzenesulfonate, sorbate, picrate, benzoate, cinnamate, and the like. Hydrochloride salts are a preferred group.

II. Cell Death Inhibiting Compositions

The invention provides pharmaceutical compositions which are effective as inhibitors of cell death, particularly apoptotic cell death, when administered in cell culture or in vivo. The inhibitors include compounds of general formula I:

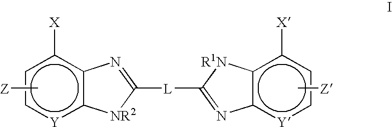

In formula I, X, X′, Z and Z′ are independently selected from the group consisting of hydrogen, lower alkyl, hydroxyl, cyano, carboxylic acid, amino, nitro, and halogen. At least one of X, X′, Z and Z′ is hydroxyl; preferably, at least one of X and X′ is hydroxyl. The linker L is CH₂ or CHCH₃, and is preferably CH₂.

The groups Y and Y′ are independently selected from nitrogen and C-Z (or C-Z′, respectively), and are preferably CH.

In the ring moieties NR¹ and NR², R¹ and R² are independently selected from hydrogen and lower alkyl. In further embodiments, NR¹ and NR² are independently selected from NH and NCH₃.

In further embodiments, X, X′, Z and Z′ are independently selected from hydrogen, methyl, hydroxyl, carboxylic acid, amino, nitro, chloro, and fluoro, where at least one of X and X′ is hydroxyl. In further embodiments, Z and Z′ are independently selected from hydrogen, hydroxyl, carboxylic acid, chloro, and fluoro. In one embodiment, the compound is 2-(benzimidazol-2′-yl)methyl-4-hydroxy benzimidazole (where L is CH₂, Y and Y′ are C—H, R¹, R² and X are hydrogen, and X′ is hydroxyl).

It should be understood that the compounds of formula I may exist in other forms depending on solvent, pH, temperature, and other variables known to practitioners skilled in the art. For example, equilibrium forms of many of the compounds may include tautomeric forms. The compounds may also be chemically modified to enhance specific biological properties, such as biological penetration, solubility, oral availability, stability, metabolism, or excretion. The compounds may also be modified to prodrug forms, such that the active moiety results from the action of metabolic or biochemical processes on the prodrug.

III. Preparation of Compounds

The compounds of formula I may be synthesized using a variety of routes known to those in the field. Some of these are described in detail in co-owned application Ser. No. 09/587,043, which is incorporated by reference. Syntheses may start with substituted benzenes (where Y is C-Z) or pyridines (where Y is nitrogen). Variously substituted benzenes and pyridines are frequently commercially available, or they may be prepared by known methods, typically employing electrophilic aromatic substitution.

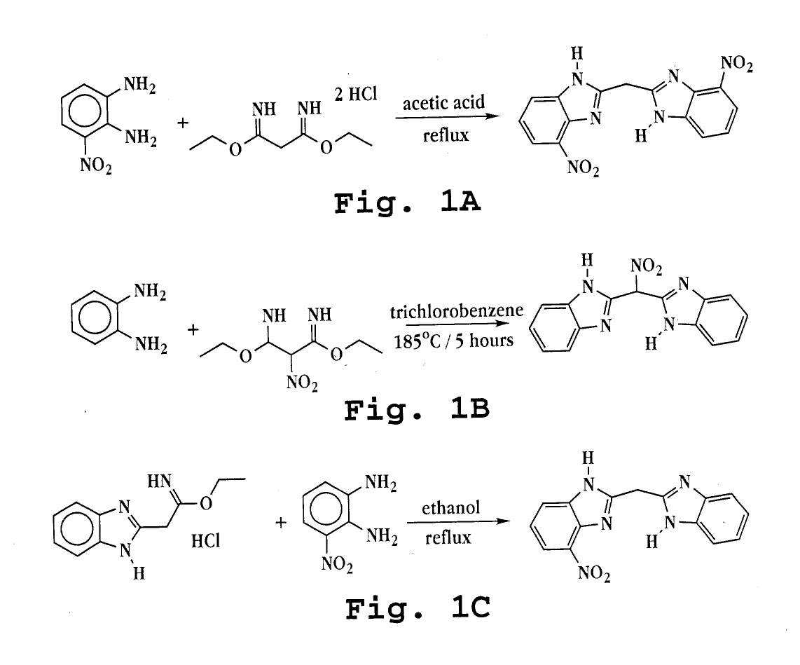

Symmetrical bis-benzimidazole compounds of formula I where L is —CH₂— may be synthesized by reacting two equivalents of the correspondingly functionalized orthodiamino benzene with one equivalent of diethyl malondiimidate. See, for example, FIG. 1A. Bis-(4-aza)benzimidazole compounds may be similarly prepared using a 2,3-diamino pyridine. Compounds where L is NH or NR¹ are prepared by a nucleophilic displacement reaction between a 2-amino- or 2-(alkylamino)-benzimidazole (or benzoxazole or benzothiazole) and a benzimidazole (or benzoxazole or benzothiazole) containing a leaving group at the 2-position, e.g. a 2-bromobenzimidazole.

Unsymmetrically substituted compounds may be synthesized, for example, by reacting one equivalent of a functionalized ortho-diamino benzene or pyridine, and one equivalent of a differently functionalized ortho-diamino benzene or pyridine, with one equivalent of diethyl malondiimidate or substituted malondiimidate. Such routes may lead to mixtures, however. An alternate route, illustrated in FIG. 1C and in Example 1 for preparation of bis-benzimidazoles, employs a benzimidazol-2-yl imidoate, prepared from the corresponding 2-cyanomethyl benzimidazole (commercially available from Aldrich). This intermediate is reacted with a substituted ortho-diaminobenzene, such as 2,3-diaminophenol in Example 1, to give the bis-benzimidazole as shown. A corresponding 2-cyanomethyl 4-aza-benzimidazole may be substituted for the benzimidazole as desired.

Compounds where the bridging carbon is substituted, e.g. with methyl, nitro, amino, or oxo (carbonyl), may be prepared using the corresponding substituted malondiimidate, e.g. as shown in FIG. 1B, in protected form if necessary. A preferred method for forming compounds in which the bridging group is a carbonyl group involves reaction of a 2-lithiated benzimidazole with a benzimidazole-2-carboxylate. Either reactant may also be derived from an 2-azabenzimidazole.

IV. Mechanisms of Cell Death

A. Distinction Between Apoptosis and Necrosis

Two distinct patterns of pathologic cell death have been described in the literature. The first pattern is consistent with necrosis, a passive process in which collapse of internal homeostasis leads to cellular dissolution (Wyllie et al., 1980a). The process involves loss of integrity of the plasma membrane and subsequent swelling, followed by lysis of the cell (Schwartz et al., 1993). This pattern manifests an early loss of membrane integrity, abnormal organellar morphology, cellular swelling, occurrence in foci, and lysosomal rupture.

The second pattern, consistent with apoptosis, occurs in scattered cells rather than in foci, and features chromatin condensation, nuclear fragmentation, decrease in cellular volume, plasma membrane blebbing, morphological preservation of organellar structure and membrane integrity, budding off of cellular fragments, and retained lysosomal contents (Wyllie et al., 1984). The observation of apoptosis is characterized by condensation of the cytoplasm and nucleus of dying cells. Ultrastructurally, the chromatin becomes electron dense, begins to accumulate at the inner surface of the nuclear envelope, and eventually fills the entire nucleus. The cell breaks up into smaller membrane bound fragments, which may contain individual organelles and remnants of the nucleus, which are rapidly phagocytosed by surrounding cells. As a result, apoptosis is not associated with a classical inflammatory response typical of other forms of cell death, such as necrosis.

Cell death in some tissues can exhibit features characteristic of both apoptosis and necrosis. In these cases, the rate of apoptosis may greatly exceed the rate of phagocytosis, such that the debris of apoptotic cells accumulates and breaks down by a process called secondary necrosis.

B. Neuronal Apoptosis

Apoptosis has been associated with ischemic injury, such as typically occurs in cases of myocardial infarction, reperfusion injury and stroke (Walton et al., 1997; MacManus et al., 1993). Apoptosis is also associated with immunoreactive and immunodegenerative states and a variety of neurodegenerative disorders, including Alzheimer's disease, ALS and motor neuron degeneration, Parkinson's disease, peripheral neuropathy, Down's syndrome, age related macular degeneration (ARMD), Huntington's disease, spinal muscular atrophy, and HIV encephalitis.

Apoptosis has also been implicated as the primary mode of cell death in models of increased intraocular pressure (IOP) in rats and in other experimental procedures that cause retinal ganglion cell loss, including optic nerve transection in monkeys, rabbits, and rats. Recent studies on the mechanism of retinal ganglion cell death in experimental glaucoma indicate that the cells die by apoptosis (Nickells, 1996; Laquis et al., 1998).

Apoptosis can have particularly devastating consequences when it occurs pathologically in cells that do not normally regenerate, such as neurons. Because such cells are not replaced when they die, their loss can lead to debilitating and sometimes fatal dysfunction of the affected organ.

V. In Vitro Model of Apoptosis: Oxygen/Glucose Deprived Retinal Ganglion Cells

Assays for apoptotic and/or necrotic death of retinal ganglion cells are useful for selecting compounds that are efficacious in the treatment of disease conditions associated with ischemia, e.g. stroke, glaucoma and other neurodegenerative diseases. An RGC culture system, such as described in co-owned U.S. Pat. No. 6,379,882 and in copending and co-owned US application published as US 20020102597 on Aug. 1, 2002, has been established as a general in vitro model for ischemia, as a model system for specialized forms of ischemia, such as that which manifests in cerebral ischemia and in glaucoma, and for neurodegenerative diseases in general. In the in vitro model for ischemia, cell death is induced by growth factor deprivation and/or oxygen/glucose deprivation (OGD).

A. Obtaining and Culturing Retinal Ganglion Cells

Retinal ganglion cells (RGCs) are central nervous system neurons that extend their axons from the retina through the optic nerve to either the geniculate nucleus or (as in the rat) directly to the superior colliculus or optic tectum. RGCs relay visual signals from the retina to the rest of the brain. These glutamatergic neurons can be purified to almost 100% purity from either the rat or mouse retina using monoclonal antibodies against the surface protein Thy 1 by an immunopanning method, as described in Example 2. RGCs can be kept in culture for a period of four weeks or longer.

B. Methods of Detecting Cell Death in RGC's

Necrotic cell death, as described above, is characterized by loss of cell membrane integrity and permeability to dyes such as propidium iodide (PI), which binds to the DNA of cells undergoing primary and secondary necrosis (Vitale et al., 1993). Necrosis is distinguishable from apoptosis in that cell membranes remain intact in the early stages of apoptosis. A PI dye exclusion assay used in parallel with an assay for apoptosis, as described below, can thus distinguish apoptotic from necrotic cell death.

Detection of programmed cell death, or apoptosis, may be accomplished via staining with annexin V-FITC, a technique known in the art. One of the earliest events in programmed cell death is the translocation of phosphatidylserine, a membrane phospholipid, from the inner side of the plasma membrane to the outer side. Annexin V, a calcium-dependent phospholipid binding protein having a high affinity for membrane bound phosphatidylserine, can thus be used to stain cells undergoing apoptosis, with detection and quantitation of apoptotic cells by flow cytometry or any other method of fluorescent detection (Vermes et al., 1995; Walton et al., 1997).

C. Quantitation of Cell Survival

Necrotic cell death, as described above, is characterized by loss of cell membrane integrity and permeability to dyes such as propidium iodide (PI), which binds to the DNA of cells undergoing primary and secondary necrosis (Vitale et al., 1993). Necrosis is distinguishable from apoptosis in that cell membranes remain intact in the early stages of apoptosis. A PI dye exclusion assay used in parallel with an assay for apoptosis, as described below, can thus distinguish apoptotic from necrotic cell death. Detection of programmed cell death, or apoptosis, may be accomplished via staining with annexin V-FITC, a technique known in the art. One of the earliest events in programmed cell death is the translocation of phosphatidylserine, a membrane phospholipid, from the inner side of the plasma membrane to the outer side. Annexin V, a calcium-dependent phospholipid binding protein having a high affinity for membrane bound phosphatidylserine, can thus be used to stain cells undergoing apoptosis, with detection and quantitation of apoptotic cells by flow cytometry or any other method of fluorescent detection (Vermes et al., 1995; Walton et al.; 1997).

VI. In Vivo Models of Ischemia

Preferred compositions of the invention are those determined to be efficacious in increasing cell survival in in vitro oxygen/glucose-deprived RGCs, as described in Section V above, by at least 25%, preferably 40%, more preferably 75%, and most preferably 100% or more, relative to untreated control RGCs. Such compositions are further tested in established animal models for ischemia. Various in vivo models have been described that mimic the symptoms of ischemia. These include the gerbil model of global ischemia, produced by transient occlusion of carotid arteries of the gerbil neck (Kirino, 1982), the rat four-vessel occlusion model for global ischemia (Pulsinelli et al., 1979), and the rat middle cerebral artery occlusion (MCAO) model of focal ischemia (Tamura et al., 1981).

Animal stroke models using focal cerebral infarction have been established in cats, dogs, primates, gerbils and rats, and are believed to be directly relevant to clinical experience. The most commonly used focal ischemia model in the rat is the right middle cerebral artery occlusion (MCAO) model developed by Koizumi and co-workers (Koizumi et al., 1986), described in Example 14 below. Briefly, the middle cerebral artery is occluded with nylon filament by insertion from the external carotid artery. The MCAO model requires no craniectomy and allows easy reperfusion.

VII. Biological Activity of Subject Compounds

A. Effect of Compounds on Oxygen/Glucose Deprived RGCs

The extent of protection of RGCs by test compounds was determined as described in Section V above and in Examples 2-3. Each compound was added to control (non-OGD) cells and to cells deprived of oxygen and glucose (OGD) for the time period from 30 minutes prior to OGD, during OGD, and for 24 and 48 hours after OGD. Untreated OGD cells were also included as controls.

Table 1 gives the value of EC₅₀ (concentration at which 50% of cells are protected from cell death relative to OGD control cells) for series of representative compounds in accordance with formula I. In Table 1, derivatives of 2,2′-methylenebisbenzimidazole (i.e. formula I where L is —CH₂— and Y and Y′ are C—H) are named by the substitution on the benzene rings of the benzimidazoles.

TABLE 1
Protection of Oxygen/Glucose Deprived RGCs

| SNX No. | EC₅₀ (nM) | Substituents on 2,2'-methylenebisbenzimidazole |
|---|---|---|
| 923 | 0.7 | 4-amino-5′-chloro |
| 940 | 3.0 | 4-amino-4′-fluoro |
| 903 | 3.2 | 4,4′-diamino |
| 977 | 5.0 | 4-amino-5′-carboxylic acid |
| 918 | 6.4 | 4-methyl |
| 917 | 10 | 5,5′-difluoro |
| 935 | 13 | 4,4′-diamino-5,5′-dichloro |
| 947 | 13 | 4-amino-5-chloro |
| 938 | 22 | 5-nitro |
| 937 | 28 | 4-nitro-5-chloro |
| 944 | 29 | 4-amino-4′-methyl |
| 912 | 31 | 4-amino |
| 936 | 32 | 4-chloro |
| 926 | 40 | 4-hydroxy |
| 934 | 84 | 4,4′-dinitro-5,5′-dichioro |
| 924 | 90 | 5,5′,6,6′-tetrachloro |
| 904 | 106 | 4,4′-dimethyl |
| 942 | 170 | 4-amino-5′-fluoro |
| 930 | 210 | 4,4′-difluoro |
| 943 | 260 | 5-amino |
| 946 | 530 | 4-amino-5′-hydroxy |
| 909 | 600 | 4,4′-dihydroxy |
| 897 | 650 | 5,5′-dinitro |
| 898 | 700 | 5,5′-diamino |
| 927 | 2200 | 5,5′-dichloro |
| 901 | 2800 | 4,4′-dinitro |
| 899 | 5300 | 5-chloro |
| 931 | 9300 | 5,5′-dicyano |

As can be seen from the data in Table 1, the compounds protected neurons from apoptotic cell death, compared to untreated control OGD cells, some at very low conentrations.

Table 2, below, shows dose-dependent data (increase in survival compared to OGD cotrol, in a similar assay) for selected bis-benzimidazole compounds of formula I. Symbols in the Table are interpreted as follows:

+ up to 50% increase

++ 50%-100% increase

+++ >100% increase

−− negligible or no increase

nd not determined

TABLE 2
Increase in Survival of OGD Cells

| Cmpd No. | | | | | | | |
|---|---|---|---|---|---|---|---|
| SNX- | 857 | 899 | 900 | 901 | 903 | 904 | 909 |
| Substitution | none | 5-Cl | 4-NO₂ | 4,4′-NO₂ | 4,4′-NH₂ | 4,4′-Me | 4,4′-OH |
| Concn, μM | Percent Increase in Survival over OGD Control Cells | | | | | | |
| 0.01 | + | + | ++ | ++ | +++ | + | + |
| 0.1 | +++ | ++ | +++ | ++ | +++ | +++ | ++ |
| 1 | ++ | ++ | ++ | +++ | +++ | +++ | +++ |
| 10 | +++ | + | + | +++ | + | −− | + |

| Cmpd. No. | | | | | | | | |
|---|---|---|---|---|---|---|---|---|
| SNX- | 912 | 923 | 926 | 929 | 930 | 931 | 897 | 898 |
| Substitution | 4-NH₂ | 4-NH₂- | 4-OH | 4,4′- | 4,4′-F | 5,5′-CN | 5,5′-NO₂ | 5,5′- |
| | | 5′-Cl | | CF₃ | | | | NH₂ |
| Concn, μM | Percent Increase in Survival over OGD Control Cells | | | | | | | |
| 0.001 | ++ | +++ | + | + | + | −− | nd | nd |
| 0.01 | ++ | +++ | ++ | ++ | +++ | + | ++ | + |
| 0.1 | ++ | +++ | +++ | ++ | + | + | ++ | + |
| 1 | +++ | +++ | +++ | + | ++ | −− | ++ | + |
| 10 | +++ | +++ | ++ | −− | ++ | ++ | nd | −− |

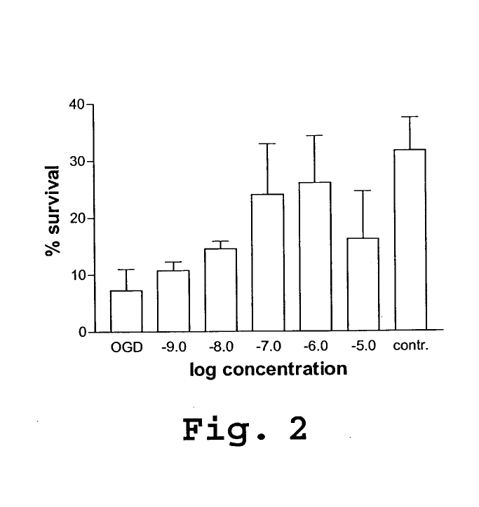

As shown in Tables 1-2, bis-benzimidazole compounds (Structure I) having substituents at one or both 4 positions, e.g. amino, nitro, methyl, trifluoromethyl, fluoro, or hydroxyl (SNX 900, 901, 903, 904, 909, 912, 923, 926, 929, and 930) were more effective overall than the unsubstituted compound, although for the methyl- and trifluoromethyl-substituted compounds, there were signs of toxic effects at higher doses (i.e. 10 μM). Bis-benzimidazole compounds having only 5 or 5,5′ substitution (e.g. the last three entries in Table 2) were found to be generally less effective than the 4-substituted counterparts. Compounds with 4-amino and 4-hydroxy substitution were particularly effective, with low toxicity. For example, the TI (therapeutic index) for the compound designated SNX-926 was >250. FIG. 2 shows dose-response data graphically for this compound, where “OGD” represents untreated OGD cells, and “contr.” represents untreated, non-OGD cells.

Structures and EC₅₀ values, as described above of additional bis-benzimidazole compounds, as well as an indole analog (SNX 1772) and a 4-aza-benzimidazole analog (SNX-911) are shown in Table 3.

TABLE 3

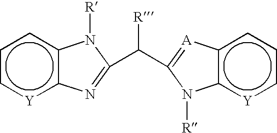

| SNX No. | R′ | R″ | R′′′ | Y | A | EC₅₀ (nM) |
|---|---|---|---|---|---|---|
| 911 | H | H | H | N | N | 2.6 |
| 925 | H | H | CH₃ | C | N | 33 |
| 952 | CH₃ | CH₃ | CH₃ | C | N | 0.4 |
| 1017 | CH₃ | H | ═O | C | N | 11 |
| 1719 | H | H | ═O | C | N | 2.0 |
| 1720 | CH₃ | CH₃ | ═O | C | N | 32 |
| 1772 | H | H | ═O | C | C | 0.9 |

VII. Methods of Treatment

In accordance with the invention, cell death is inhibited by administering, in a pharmaceutically acceptable carrier, a compound represented by formula I, discussed above, or pharmaceutically acceptable salts. Preferred compounds are also discussed above, and particularly include those giving EC₅₀ values, for the assay represented in Table 1, of 1 μM or less, preferably about 500 nm or less, more preferably about 100 nm or less, and most preferably about 50 nm or less.

The compositions may be used for the treatment of diseases that involve apoptotic cell death or other forms of interventional cell death. The method of treatment, dosage level, paradigm of administration, etc., may be selected from conventional methods and techniques. For example, a compound of this invention may be administered with a pharmaceutically acceptable adjuvant to a patient suffering from a disease or disorder resulting from sudden and/or pathological cell death. The compound is administered, in combination with an acceptable adjuvant or carrier, in an amount effective to lessen the severity of the disease as a result of decreasing the biological cell death.

The compounds of formula I may be used alone or in combination, and they may be combined with other classes of cell death-inhibiting compounds, to increase the effect of therapy, or as a prophylaxis to decrease the progression of a cell death-induced disease. The compounds of this invention may also be used in combination with other therapeutic agents, including anti-hypertensive agents, antibiotics, immunomodulators or anti-inflammatory agents. In combination therapy, the compounds may be administered either sequentially or concurrently.

Pharmaceutical compositions of this invention comprise any of the compounds of formula I and their pharmaceutically acceptable salts, together with pharmaceutically acceptable carriers, adjuvants or vehicles. The pharmaceutical compositions may be administered orally, parenterally (which includes subcutaneous, intravenous, intramuscular, intra-articular, intracutaneous, intrasynovial, intrastemal, intrathecal, epidural, intralesional, intracerebroventricular, or intracranial), by inhalation spray, topically, rectally, nasally, buccally, vaginally, or via an implanted reservoir. Injectable preparations include a sterile injectable aqueous or oil composition or a suspension. For treatment or prevention of damage resulting from therapeutic intervention in cardiac cells, e.g. during arterial graft or angioplasty, the compounds may be administered locally, e.g. by catheter or stent, to the affected artery.

For reducing post-ischemic damage, the compounds may be administered via ICV administration at levels of approximately 5-25 mg/kg. For IV administration, a more convenient but less efficient route, higher doses, e.g. 25-50 mg/kg, are advised. Appropriate dosages of various compounds of formula I may be higher or lower, depending on the potency of the particular compound. Relative potencies of a variety of compounds are given above, and others may be determined in assays as described above and in the Examples below. As always, optimum dosages in human therapy will vary according to factors such as the route of administration, the age of the patient, other existing medical conditions, and the type and severity of symptoms, and may be determined according to standard methods known to skilled practitioners.

VIII. Indications

Cell death-mediated conditions which may be treated or prevented by the compositions of the invention include ischemic injury, such as stroke or myocardial infarction, ischemic diseases, inflammatory diseases, trauma, including myocardial damage, autoimmune diseases, and neurodegenerative diseases.

Ischemic damage to the central nervous system (CNS) may result from either global or focal ischemic conditions. Global ischemia occurs under conditions in which blood flow to the entire brain ceases for a period of time, such as may result from cardiac arrest. Focal ischemia occurs under conditions in which a portion of the brain is deprived of its normal blood supply, such as may result from thromboembolytic occlusion of a cerebral vessel, traumatic head injury, edema, and brain tumors. Ischemic diseases include cerebral ischemia, such as results from stroke, myocardial infarction, retinal ischemia, macular degeneration, and glaucoma.

Various neurodegenerative diseases which may involve apoptotic cell death include Alzheimer's disease (Kim et al., 1997), ALS and motor neuron degeneration (Greenlund et al., 1995), Parkinson's disease (Ghosh et al., 1994), peripheral neuropathies (Batistatou et al., 1993), Down's syndrome (Busciglio et al., 1995), age related macular degeneration (ARMD) (Hinton et al., 1998), Huntington's disease (Goldberg et al., 1996), spinal muscular atrophy (Liston et al., 1996), and HIV encephalitis (Lazdins et al., 1997).

Although the invention has been described with respect to particular treatment methods and composition, it will be apparent to those skilled that various changes and modifications can be made without departing from the invention.

### EXAMPLES

The following examples illustrate but are not intended in any way to limit the invention.

### Example 1

### Preparation of 2-(benzimidazol-2-ylmethyl)benzimidazol-7-ol dihydrochloride (SNX-926)

A. Synthesis of 2-benzimidazol-2-yl- 1 -ethoxyethanimine dihydrochloride (Ethyl imidate of 2-benzimidazolylacetonitrile)

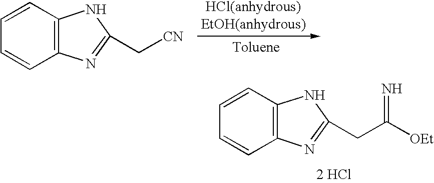

To a 3-neck round-bottom flask equipped with a magnetic stirbar and a thermometer was added 2-benzimidazolylacetonitrile (10.0 g, 0.064 mol). Anhydrous toluene (300 mL) was charged to the flask via cannula. The tan slurry was cooled to ˜0° C. using an ice bath. Anhydrous ethanol (9.5 mL) was added to the slurry via syringe. Anhydrous HCl (g) was bubbled slowly through the solution for approximately 1 hour. HCl addition was ended and the slurry was allowed to slowly warm to ambient temperature with stirring overnight. Anhydrous diethyl ether (300 mL) was charged to the slurry slowly. The slurry was filtered and vacuum dried to afford an off-white powder, 2-benzimidazol-2-yl-1-ethoxyethanimine dihydrochloride (17.2 g, 0.062 mol), in 97.7% yield. The solid was used directly in the second step of the reaction, below.

B. Cyclocondensation with 2,3-diaminophenol

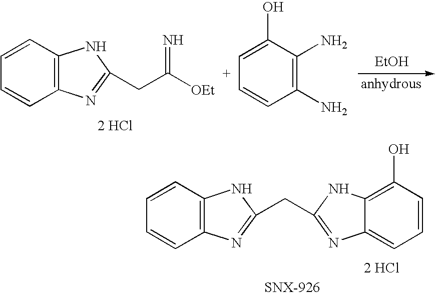

To a 3-neck round bottom flask equipped with a magnetic stirbar, reflux condenser, and heating mantle was charged 2-benzimidazol-2-yl-1-ethoxyethanimine dihydrochloride (imidate), as prepared above (17.2 g, 0.062 mol.) Anhydrous ethanol (450 mL) was charged to the imidate with stirring to give a yellow slurry. To a separate 3-neck round bottom flask was charged 2,3-diaminophenol (7.5 g, 0.060 mol.) (If desired, the 2,3-diaminophenol can be purified prior to use using the following procedure: Dissolve in ˜30 volumes of DI water, adjust the pH of the solution to ˜2 with HCl, filter the solution to remove fine particulates (impurities), then remove solvent from the filtrate and vacuum dry.)

Anhydrous ethanol (225 mL) was charged via cannula to the phenol. The slurry was warmed to ˜35° C. via heating mantle while the phenol slurry was heated to reflux. The refluxing solution containing the phenol was charged to the imidate slurry via cannula. The combined reaction mixture was brought to reflux temperature and allowed to reflux gently overnight under an atmosphere of nitrogen. The resultant dark slurry was cooled to ambient temperature, and solids were collected by vacuum filtration. The solids were washed with ˜100 mL ethanol and then vacuum dried to afford a gray solid, (17.6 g, 0.052 mol) 2-(benzimidazol-2-ylmethyl)benzimidazol-7-ol dihydrochloride. (SNX-926) in 87.1% yield.^{1,2} Data: MS: [M+1] 266.1; % C: 53.32, % H: 4.22, % N: 16.37, % Cl: 20.98; ¹H NMR (400 MHz, d₆-DMSO) δ10.9 (broad, 1H), 7.8 (dd, 2H), 7.5 (m, 2H), 7.3 (dd, 1H), 7.2 (s, 1H), 6.9 (dd, 1H), 5.1 (broad s, 2H).

A small amount of a benzoxazole side product (below) may be formed. This compound can be removed as follows: Add ˜20 volumes of ethanol and 0.6 volumes of 6M HCl to the solid product, allow the resulting slurry to reflux overnight, cool to ambient temperature, collect the solids by vacuum filtration, and dry under vacuum.

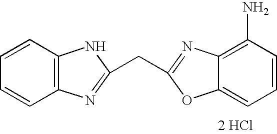

### Example 2

### Purification and Culture of Retinal Ganglion Cells (RGC's)

RGCs from postnatal day 8 (P8) Sprague-Dawley rats were purified as previously described (Barres et al., 1988; Meyer-Franke et al., 1995). Purified retinal ganglion cells were plated onto tissue culture plastic precoated with poly-D-lysine and merosin, and cultured in serum-free Neurobasal medium (Gibco) containing various supplements.

A. Isolation of RGC's

The tissue from P8 Sprague/Dawley rat retinas (Simonsen Labs, CA) was dissociated enzymatically to obtain a suspension of single cells, by incubating the tissue in a papain solution (15 U/ml per retina, Worthington) in Earle's balanced salt solution (EBSS, Gibco) containing L-cysteine at 37° C. for an appropriate time to dissociate the tissue. The tissue was then disrupted sequentially with a 1 ml pipette, in a solution containing ovomucoid (Boehringer-Mannheim), DNase (Sigma), and bovine serum albumin (BSA; Sigma) to yield a single cell suspension. The cells were then washed in a suspension of ovomucoid/BSA.

B. Panning Procedure

Using sequential immunopanning, RGCs can be purified to greater than 99% homogeneity. Typically, 20-30% of the RGCs are isolated, which represents about 40,000 to 60,000 RGCs per P8 (post-natal, day 8) animal.

Panning plates were prepared in petri dishes (150 mm for the anti-rabbit IgG plates and 100 mm for the T11D7 plate) by incubating with Tris buffer solution (pH 9.5) containing 10 mg/ml of secondary antibody for approximately 12 hours at 4° C. Either affinity-purified goat anti-rabbit IgG (H+L chain-specific; Jackson Laboratories) or affinity-purified goat anti-mouse IgM (mu chain-specific; Jackson Laboratories) was used as the secondary antibody. The plates were then washed three times with phosphate-buffered saline (PBS), and the dish with anti-mouse IgM antibodies was further incubated with Thy 1.1 IgM monoclonal supernatant (antibody against mouse Thy 1.1, T11D7e2, ATCC, TIB 103) for approximately 2 hours at room temperature. After removing the supernatant, the plate was washed three times with PBS. To prevent non-specific binding of cells to the panning dish, PBS containing 2 mg/ml bovine serum albumin (BSA) was placed on the panning dishes.

The retinal cell suspension was incubated in anti-rat macrophage antiserum (Axell) for approximately 20 minutes, centrifuged, resuspended in PBS and incubated on an anti-rabbit panning plate for approximately 45 minutes. The plate was gently swirled every 15 minutes to ensure access of all cells to the surface of the plate. Following this, the cell suspension was transferred to a second anti-rabbit panning plate for approximately 30 minutes. Non-adherent cells were removed with the supernatant, filtered through a 15 μm Nytex mesh (Tetko) and placed on the T11D7 panning plate. After approximately 45 minutes, the plates were washed eight times with PBS to remove the non-adherent cells.

C. Removal of Adherent Cells

A trypsin solution (0.125%) was prepared by diluting a trypsin stock (Sigma) in EBSS (Ca²⁺ and Mg²⁺ free Eagle's balanced salt solution). The cells in the panning dish were incubated with 4 ml of this solution for ten minutes in a 5% CO₂ incubator. The cells were dislodged by gently pipetting the trypsin solution around the plate. Ten ml of 25% fetal calf serum medium was added to inactivate the trypsin, and the cells were centrifuged and resuspended in culture medium.

D. Culturing of RGC's

Approximately 5,000 purified RGCs were cultured in 96-well plates (Falcon), precoated with poly-D-lysine (PDL, 70 kD, 10 mg/ml; Sigma) and merosin (2 mg/ml; Gibco). The RGCs were cultured in serum-free Neurobasal medium (Brewer et al., 1993; Gibco) containing Sato-Bottenstein and B27 (Gibco) supplement, insulin (Sigma, 5 mg/ml), brain-derived neurotrophic factor (BDNF, 25 ng/ml; Preprotech), ciliary neurotrophic factor (CNTF, 20 ng/ml; Preprotech) and forskolin (10 mM, Sigma). The percentage of surviving cells was assessed at 3, 7, and 14 days by the MTT assay.

### Example 3

### Oxygen/Glucose Deprivation (OGD) Model for Ischemia

Retinal ganglion cells were grown in 96-well plates for 5 days in serum-free medium as described above. On the sixth day cells were washed three times in a salt solution, e.g. Earle's balanced salt solution (EBSS, Gibco), containing glucose for control cells, and lacking glucose for test cells (oxygen/glucose-deprived cells). Control cells were further incubated in a 5% CO₂ incubator while OGD cells were deprived of oxygen in an anaerobic chamber (for 3 hours). Test compounds were added to control cells and OGD cells for the time period from 30 minutes prior to OGD, during OGD, and for 24 and 48 hours after OGD.

After 3 hours OGD, control and test cells were transferred to growth medium with glucose and cultured an additional 48 hours in a 5% CO₂ incubator, followed by a determination of cell viability using MTT, propidium iodide and annexin assays.

For the cell viability assay, MTT was added to culture and incubated at 37° C. for 1 hr. Viable cells with active mitochondria cleave the tetrazolium ring to form a visible dark blue formazan product. Viable and dead cells are counted by bright field microscopy at various times, e.g. 24, 48, or 72 hours after oxygen/glucose and/or growth factor deprivation. All values are reported as the mean (average) +/− the standard error of the mean (SEM) for at least three replicate cultures.

24 hours after oxygen/glucose deprivation (OGD), approximately 25% fewer retinal ganglion cells were determined to be alive relative to non-deprived control cells. After 48 hours, 40% fewer cells survived relative to non-deprived control cells. The dead cells showed the typical shrunken morphology of apoptotic cells. To confirm that the retinal ganglion cells died of programmed cell death (apoptosis) following OGD, cell cultures were labeled with FITC-coupled annexin V (ApoAlert Kit, Clonetech) and PI at 24 and 48 hours after OGD, followed by light and fluorescent microscopy. 200 cells were counted per triplicate value. The percentage of annexin positive cells was consistent with that of dead cells observed in previous experiments. Approximately 80% total dead RGCs were also annexin V positive at both 24 and 48 hours, indicating that the majority of cells died by apoptosis.

### Example 4

### In Vivo Focal Ischemia Model

A. Rat Filament Model

Adult male Wistar rats weighing 310-380 g were used. Animals were fasted overnight but allowed free access to water. Anesthesia was induced and maintained with 3% isoflurane in 0.8% oxygen. Systemic blood pressure was recorded before, during and after middle cerebral artery occlusion (MCAO) and immediately before administering the test compound. Subjects received test compound SNX 912, 5 mg/kg ICV pre-MCAO, or 25 mg/kg IV immediately following reperfusion, after two hours MCAO, as compared to control subjects, which received deionized water. Temperature was controlled and recorded before, during and following reperfusion. After reperfusion, temperature was measured every hour for 4 hrs post-reperfusion.

All animals were subjected to 2 hr of MCAO using the intraluminal filament technique of Koizume et al. (1986) as modified by Zhao et al. (1994). A midline surgical incision was made to expose the right common, external and internal carotid arteries. The common cartotid, external carotid and occipital arteries were tightly ligated, and the internal carotid artery was temporarily closed with a microvascular clip. A small incision was made in the common carotid artery and a nylon monofilament was inserted into the internal carotid artery through the common carotid artery. The filament was then carefully advanced 19 mm cephalad to occlude the middle cerebral artery at its site of origin within the Circle of Willis. Anesthesia was terminated, and upon awakening the animals were observed for the appearance of neurological deficits during MCAO. After 2 hr of MCAO, the animals were re-anesthetized with 1.5% halothane, and the occlude filament was withdrawn to allow reperfusion.

Because MCAO by the intraluminal filament technique can give rise to intra- and post-ischemia hyperthermia, rectal temperature was controlled by external heating and cooling for 6 hrs after initiating MCAO. Rectal temperature was maintained at 37.5+/−0.5° C.

B. Evaluation of Ischemic Damage Following MCAO

Animals were killed 24 hr post-reperfusion by CO₂ asphyxiation. Following asphyxiation, the brains were quickly removed and chilled in ice cold 0.9% saline for 10 min. To visualize the extent of ischemic damage, seven 2 mm thick coronal slices were cut from each brain with a tissue slicer beginning with 1 mm posterior to the anterior pole. The slices were immersed in a 0.9% saline solution containing 1.0% 2,3,5-tripheyltetrazolim chloride (TTC) and incubated at 37° C. for 30 minutes, and observed for the presence of formazan (red), which is produced by the reduction of TTC by endogenous dehydrogenase activity in normal living tissues.

It is claimed:

1. A pharmaceutical composition useful for inhibiting cell death, comprising an effective amount of a compound of formula I, or a pharmaceutically acceptable salt thereof, in a pharmaceutically acceptable carrier: 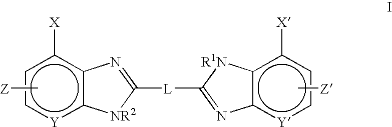
where
X, X′, Z and Z′ are independently selected from the group consisting of hydrogen, lower alkyl, hydroxyl, cyano, carboxylic acid, amino, nitro, and halogen, wherein at least one of X, X′, Z and Z′ is hydroxyl;
R¹ and R² are independently selected from hydrogen and lower alkyl;
L is —CH₂— or —CHCH₃—;
Y is selected from C-Z and nitrogen, and Y′ is selected from C-Z′ and nitrogen.

2. The composition of claim 1, wherein L is CH₂.

3. The composition of claim 1, wherein R¹ and R² are independently selected from hydrogen and methyl.

4. The composition of claim of claim 3, where R¹ and R² are hydrogen.

5. The composition of claim 4, wherein each of Y and Y′ is C—H.

6. The composition of claim 1, wherein X, X′, Z and Z′ are independently selected from the group consisting of hydrogen, methyl, hydroxyl, cyano, carboxylic acid, amino, nitro, fluoro, and chloro, wherein at least one of X, X′, Z and Z′ is hydroxyl.

7. The composition of claim 6, wherein at least one of X and X′ is hydroxyl.

8. The composition of claim 1, wherein X is hydrogen and X′ is hydroxyl.

9. The composition of claim 5, wherein X is hydrogen and X′ is hydroxyl.

10. The compound 2-(benzimidazol-2′-yl)methyl-4-hydroxy benzimidazole (designated herein as SNX-926).

11. A method of inhibiting cell death, comprising administering to a subject in need of such treatment an effective amount of a compound of formula I, or a pharmaceutically acceptable salt thereof, in a pharmaceutically acceptable carrier: 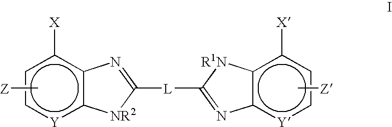
where
X, X′, Z and Z′ are independently selected from the group consisting of hydrogen, lower alkyl, hydroxyl, cyano, carboxylic acid, amino, nitro, and halogen, wherein at least one of X, X′, Z and Z′ is hydroxyl;
R¹ and R² are independently selected from hydrogen and lower alkyl;
L is —CH₂— or —CHCH₃—;
Y is selected from C-Z and nitrogen, and Y′ is selected from C-Z′ and nitrogen.

12. The method of claim 11, wherein L is CH₂.

13. The method of claim 11, wherein R¹ and R² are independently selected from hydrogen and methyl.

14. The method of claim 13, wherein R¹ and R² are hydrogen.

15. The method of claim 11, wherein Y and Y′ are C-Z and C-Z′, respectively.

16. The method of claim 15, wherein each of Y and Y′ is C—H.

17. The method of claim 11, wherein at least one of X and X′ is hydroxyl.

18. The method of claim 17, wherein X is hydrogen and X′ is hydroxyl.
